(19) United States
(12) Patent Application Publication
(10) Pub. No.: US 2023/0248559 A1
(43) Pub. Date: Aug. 10, 2023

(54) DEVICES, SYSTEMS, AND METHODS RESISTING MIGRATION AND METHOD OF IMPLANTATION THEREOF

(71) Applicant: BOSTON SCIENTIFIC SCIMED, INC., Maple Grove, MN (US)

(72) Inventors: Peter L. Dayton, Brookline, MA (US); John Thomas Favreau, Shrewsbury, MA (US); Darren Gerard Curran, Galway (IE); John O'Driscoll, Galway (IE); Kevin Wood, Marlborough, MA (US); Douglas Melanson, Natick, MA (US); Marc A. Barthet, Marseille (FR); Jean-Michel Gonzalez, Fuveau (FR)

(73) Assignee: BOSTON SCIENTIFIC SCIMED, INC., Maple Grove, MN (US)

(21) Appl. No.: 18/106,884

(22) Filed: Feb. 7, 2023

Related U.S. Application Data
(60) Provisional application No. 63/307,771, filed on Feb. 8, 2022.

(51) Int. Cl. A61F 5/00

(52) U.S. Cl. CPC A61F 5/0076

(57) ABSTRACT

An implantable device configured to be deployed at a deployment site such as across a body passage. The implantable device has features enhancing the resistance of at least a portion of the implantable device to migration from the deployment site. The implantable device may have surfaces contacting tissue at the deployment site which are configured to promote tissue ingrowth with respect to the implantable device. Surfaces of the implantable device not contacting tissue may be enlarged and coated with a coating material increasing the strength of the coated portions of the implantable device to resist migration of the implantable device with respect to the deployment site.

### CROSS-REFERENCE TO RELATED APPLICATIONS

This application claims the benefit of priority of U.S. Provisional Application No. 63/307,771, filed Feb. 8, 2022, the entire disclosure of which is hereby incorporated by reference herein for all purposes.

### FIELD

The present disclosure relates generally to the field of implantable devices, systems, and methods. More particularly, the present disclosure relates to the field of devices, systems, and methods for resisting migration of an implantable device from an anatomical passage in which the device has been deployed.

### BACKGROUND

Various medical treatments involve occluding flow of materials through a body passage. For instance, treatment methods for various medical conditions, such as obesity, diabetes, or duodenal ulcers, involve bypassing the duodenum or restricting flow of materials through the duodenum. Common bariatric procedures include Sleeve Gastrectomy, Roux-en-Y Gastric Bypass and Gastric Banding, and are either obstructive (as in Gastric Banding and Sleeve Gastrectomy), or malabsorptive or both (as with Roux-en-Y). All of these procedures are highly invasive with the associated possibility of serious complications. A newer, less invasive procedure, with less associated trauma and lower risk for complication, involves creation of an anastomosis, such as between the stomach and the jejunum (a gastrojejunostomy, or GJ for short), and a pyloric closure via an endoscopic procedure (e.g., endoscopic ultrasound procedure, or EUS for short), such as a NOTES (natural orifice transluminal endoscopic surgery) procedure. If the treatment requires complete bypass of the duodenum, then implantable (e.g., full implantable) of the pylorus may be indicated. An implantable device, such as a duodenal exclusion device, may be placed in the pyloric sphincter to inhibit or block passage of materials (fluid, chyme, etc.) from the stomach through the pylorus and into the duodenum. Various challenges to preventing migration of a deployed implantable device are presented by the natural movements of the body (e.g., the gastrointestinal system) as well as the constant flow of materials against the implantable device. Peristaltic movement of the pylorus to pass materials therethrough (e.g., distally into the small intestine), generally less frequent reverse peristalsis through the pylorus (proximally into the stomach), as well as the natural tendency of the pylorus to eject materials therein present particular challenges for placement and retention of pyloric implantable devices. Backpressure from the large and small intestines, via the duodenum, also may cause migration of the device. Improved structures and methods for increasing resistance of implantable devices to migration would be welcome.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, an implantable device, extending along a longitudinal axis and having a first end, a second end, and an intermediate region therebetween, includes a saddle along the intermediate region, a first retention member extending radially outwardly from the longitudinal axis along the first end, and a second retention member extending radially outwardly from the longitudinal axis along the second end, where: at least a portion of the surface of the saddle and at least a portion of inwardly-facing surfaces of the first retention member and the second retention member facing towards the saddle are configured to promote tissue ingrowth therewith, and outwardly-facing sides of the first retention member and the second retention member facing away from the saddle have sufficiently large surface areas such that coating material provided thereon increases the pull out strength of the first retention member and the second retention member.

In some embodiments, the outwardly-facing side of at least one of the first retention member or the second retention member has a surface area of greater than about 500 mm².

In some embodiments, a lip extends axially from the second retention member a greater distance than the distance between the inwardly-facing side and the outwardly-facing side of the second retention member and is configured to promote tissue growth therein.

In some embodiments, the first retention member and the second retention member each are double wall retention members with an inner wall along the inwardly-facing side thereof and an outer wall along the outwardly-facing side thereof; and the distance between the inner wall and the outer wall of the second retention member is greater than the distance between the inner wall and the outer wall of the first retention member.

In some embodiments, the implantable device further includes a lip axially extending from at least one of said first retention member or said second retention member in a direction away from said saddle. In some embodiments, the lip is coated with a coating material reducing flexibility thereof.

In some embodiments, the first retention member and the second retention member are different sizes, shapes, configurations, and/or dimensions.

In some embodiments, the saddle is configured to be positioned through a pylorus; the first retention member is configured to be seated in a portion of a stomach surrounding the pylorus; and the second retention member is configured to extend into a duodenum extending from the pylorus. In some embodiments, the saddle is narrowed to occlude flow of materials through the pylorus.

In some embodiments, the diameter of the first retention member is greater than the diameter of the second retention member.

In some embodiments, the saddle, and sides of the first retention member and the second retention member facing inwardly towards the saddle have walls with interstices defined therein and are left uncoated to promote tissue ingrowth into the interstices.

In accordance with various principles of the present disclosure, an implantable device is formed from a tubular element having a first end, a second end, and an intermediate region therebetween, and a lumen defined therethrough. In some embodiments, the implantable device includes a saddle along the intermediate region; a first retention member defined by a portion of the tubular element along the first end of the tubular element having a first diameter larger than the outer diameter of the saddle to form a first retention member inner wall facing the saddle and a first retention member outer wall facing away from the saddle, and a second retention member defined by a portion of the tubular element along the second end of the tubular element having a second diameter larger than the outer diameter of the saddle to form a second retention member inner wall facing the saddle and a second retention member outer wall facing away from the saddle, where: the first free end of the tubular element has a diameter sufficiently smaller than the outer diameter of the first retention member to provide sufficient surface area to receive a coating material to increase the rigidity of the first retention member outer wall.

In some embodiments, the diameter of at least one of the first free end of the tubular element and the second free end of the tubular element is smaller than the largest diameter of the saddle.

In some embodiments, the retention members have different sizes, shapes, configurations, and/or dimensions.

In some embodiments, the outer diameter of the first retention member is larger than the outer diameter of the second retention member.

In some embodiments, the distance between the second retention member inner wall and the second retention member outer wall is greater than the distance between the first retention member inner wall and the first retention member outer wall.

In some embodiments, the implantable device includes a lip extending axially from the second retention member away from the saddle, and having a length greater than the distance between the second retention member inner wall and the second retention member outer wall. In some embodiments, the second retention member outer wall is coated and the outer surface of the lip is configured to promote tissue ingrowth.

In accordance with various principles of the present disclosure, a method of forming an implantable device from a tubular element having a first end, a second end, an intermediate region therebetween, and a lumen defined therethrough, includes narrowing an intermediate region of the tubular element to form a saddle along the intermediate region; increasing the diameter of the tubular element along the first end of the tubular element to be larger than the outer diameter of the saddle to form a first retention member inner wall facing the saddle and a first retention member outer wall facing away from the saddle; increasing the diameter of a portion of the tubular element along the second end of the tubular element to be larger than the outer diameter of the saddle to form a second retention member inner wall facing the saddle and a second retention member outer wall facing away from the saddle; and drawing the first free end of the tubular element inwards to have has a diameter sufficiently smaller than the outer diameter of the first retention member to provide sufficient surface area to receive a coating material to increase the rigidity of the first retention member outer wall.

In some embodiments, the first free end of the tubular element is drawn inwards to have a diameter smaller than the largest diameter of the saddle.

In some embodiments, the retention members are formed to have different sizes, shapes, configurations, and/or dimensions.

In some embodiments, the diameter of the portion of the tubular element along the first end of the tubular element is increased so that the outer diameter of the first retention member is larger than the outer diameter of the second retention member.

In some embodiments, the second retention member inner wall and the second retention member outer wall are formed at a greater from each other than the distance between the first retention member inner wall and the first retention member outer wall.

In some embodiments, a lip is formed extending axially from the second retention member away from the saddle, and having a length greater than the distance between the second retention member inner wall and the second retention member outer wall.

In some embodiments, the second retention member outer wall is coated, and the outer surface of the lip is configured to promote tissue ingrowth.

In accordance with various principles of the present disclosure, a method of deploying an implantable device at a deployment site includes performing endoscopic mucosal resection of a portion of tissue at the deployment site so that tissue growth is promoted at the resected tissue site; and deploying the implantable device with a surface contacting the resected tissue site configured to allow tissue ingrowth therein.

In some embodiments, the method includes ablating tissue at the deployment site surrounding the resected tissue using mucosal abrasion.

In some embodiments, the first retention member has an outer diameter greater than the outer diameter of the second retention member and has an inwardly-facing side contacting the tissue surrounding the inlet and configured to promote tissue ingrowth therein, and an outwardly-facing side facing away from the body passage and not contacting tissue and coated with a material increasing the pull out strength thereof, the outer diameter of the first retention member and the material coated thereon increasing the retention strength of the first retention member against migration of the implantable device into the body passage.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an embodiment of an implantable device formed in accordance with various aspects of the present disclosure and positioned in a schematic representation of a gastrointestinal environment.

FIG. 2 illustrates an elevational view of an implantable device formed as in FIG. 1.

FIG. 3 illustrates a perspective view of an implantable device formed as in FIG. 2.

FIG. 4 illustrates an elevational view of another example of an embodiment of an implantable device formed in accordance with various principles of the present disclosure.

FIG. 5 illustrates a perspective view of an implantable device formed as in FIG. 4.

FIG. 6 illustrates an elevational view of another example of an embodiment of an implantable device formed in accordance with various principles of the present disclosure.

FIG. 7 illustrates a perspective view of an implantable device formed as in FIG. 6.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “free end” of an element is a terminal end at which such element does not extend beyond.

In accordance with various principles of the present disclosure, an implantable device is sized, shaped, configured, and/or dimensioned to resist migration from the site at which the implantable device is deployed. Such site is referenced herein as a deployment site for the sake of convenience and without intent to limit, and, as used herein, is a site or location at which the device is deployed, positioned, implanted, extended, placed, etc. (such terms being used interchangeably herein without intent to limit) within a human body, and may also be the intended treatment site or position of the device once deployed and in use. The deployment site may be a body passage or lumen, such terms being used interchangeably herein without intent to limit, the broad principles of the present disclosure being applicable to various shapes and sizes of body passages, lumens, cavities, etc., or other anatomical structures. The body passage may extend between anatomical structures (e.g., a body cavity or organ adjacent the body passage) with a diameter generally larger than the body passage.

An implantable device formed in accordance with various principles of the present disclosure may be deployed with a saddle region of the device extending across, through, within, etc. (such terms being used interchangeably herein without intent to limit) a body passage or body tissue. It will be appreciated that reference to a body passage herein includes a passage formed through tissue by a medical procedure (i.e., not a naturally occurring body passage), such as a passage formed through apposed tissue walls (e.g., to form an anastomosis between the walls, or to simply hold the walls in apposition). The body passage may have an inlet and an outlet, and the implantable device may be provided with retention members (which may be alternatively referenced as flanges) configured to engage one or more anatomical structures at the deployment site to inhibit movement of the implantable device with respect to the deployment site. In some embodiments, outer walls of the retention members may be seated with respect to an anatomical structure (e.g., a body wall), such as along or adjacent the inlet and/or outlet of the body passage through which the implantable device is deployed, as anti-migration structures configured to resist migration of the implantable device with respect to the deployment site (e.g., with respect to the body passage). It will be appreciated that terms such as resist, inhibit, prevent (and other grammatical forms thereof) may be used interchangeably herein without intent to limit. The retention members may extend radially outward from along either end of the intermediate region of the implantable device (along which the saddle is positioned). It will be appreciated that terms such as along or at or on or adjacent an end may be used interchangeably herein without intent to limit unless otherwise stated, and are intended to indicate a general relative spatial relation rather than a precisely limited location. For instance, a retention member may be in the form of a lateral extension or flange wider than the intermediate region of the device and wider than a body passage in which the intermediate region of the device is deployed. The retention members are generally wider than the saddle (in a radial direction transverse to the longitudinal axis of the body passage), and are configured to seat against a body wall extending radially outwardly from the body passage.

In some embodiments, at least one of the retention members is configured to resist migration of the implantable device with respect to the deployment site. In some embodiments, the first retention member and the second retention members are shaped similarly, although such shapes may not have the same dimensions (i.e., the shapes may have different relative dimensions, scales, or proportions). For instance, the first retention member and the second retention member may be substantial duplicates of each other (optionally with different relative proportions), oriented in generally the same direction. Alternatively, in some embodiments, the first retention member and the second retention member may be mirror images (e.g., similar shapes facing in generally opposite directions, optionally with different relative proportions). In some embodiments, the first retention member and the second retention member are not generally the same or symmetrical. For instance, the first retention member and the second retention member may have different shapes, dimensions, relative proportions, etc. The outer diameter of at least one of the retention members may be increased to be significantly (e.g., about 10 times or about 15 times or about 17 times or even about 20 times, including all increments therebetween) greater than the outer diameter of the saddle to enhance retention strength to prevent migration of the implantable device with respect to the deployment site. In some embodiments, an implantable device is configured to be positioned across a pylorus, and may have an enlarged gastric retention member (e.g., with a diameter larger than the duodenal retention member) to resist distal migration from the stomach into the duodenum. For instance, whereas the duodenal retention member may have an outer diameter of about 25-34 mm (generally limited by the average internal diameter of the duodenum), the gastric retention member has more room to expand within the stomach, and may have a diameter greater than 30 mm, such as about 35 mm or about 40 mm or about 50 mm or about 60 mm, and potentially larger (such measurement encompassing increments of about 1 mm therebetween). The length of a device positioned across a pylorus may be at least about 2 mm or about 3 mm, and may be as long as about 12.5 mm, or even about 20 mm (e.g., if configured to extend across the duodenal bulb) or even about 50 mm (e.g., if configured to extend across the length of the duodenal section of the small intestines). Additionally or alternatively, a device positioned across a pylorus may have a duodenal retention member elongated (along the longitudinal axis of the device) to extend into the duodenum and to provide additional surface area for tissue ingrowth into the duodenal retention member. In some embodiments, the size, shape, configuration, and/or dimensions of at least one of the retention members are modified to enhance resistance by the retention members to migration.

In accordance with various principles of the present disclosure, one or both of the retention members are double wall retention members with two walls adjacent and in contact or spaced apart from each other, each wall forming a side of the retention member. For the sake of convenience and without intent to limit, the wall of each double-wall retention member closer to the saddle may be referenced herein as the inner side or inner wall of the retention member, and the wall of each double-wall retention member further from the saddle may be referenced herein as the outer side or outer wall of the retention member. Various aspects of the inner and outer walls of a double wall retention member formed in accordance with various principles of the present disclosure may be modified as described herein to increase the ability of the retention member to retain the implantable device in place at the deployment site.

In some aspects, the implantable device is configured to promote tissue ingrowth with respect to portions of the implantable device so that the implantable device will resist migration with respect to such tissue. In some embodiments, regions of the retention members are configured to promote tissue ingrowth with respect to such region of the implantable device. For instance, regions of the retention members may be left as uncoated regions (not treated with a substance which inhibits tissue ingrowth). It will be appreciated that reference may be made herein to terms such as regions, surfaces, areas, portions, segments, sections, etc. interchangeably and without intent to limit. The uncoated regions of the implantable device generally include tissue-contacting surfaces of the implantable device. For instance, at least a portion of the intermediate region of the device extending through a body passage and/or at least portions of inwardly facing surfaces (facing towards the intermediate region) of retention members on the device (which regions or surfaces generally are in contact with tissue at the deployment site) may be at least partially uncoated. In some embodiments, most or all tissue-contacting surfaces of an implantable device are not coated so that tissue ingrowth therein is permitted, and even promoted (e.g., by the structure or configuration of the wall of the implantable device such as described in further detail below), to inhibit or prevent migration of the implantable device with respect to the deployment site.

It will be appreciated that various coatings may be applied to implantable devices for various purposes. The coatings may be lubricious to facilitate movement with respect to the anatomy of the patient in which the device is implanted and/or to facilitate passage of materials (e.g., fluids, particles, tools, instruments, devices, etc.) therethrough and/or to prevent tissue ingrowth with respect to the implantable device (e.g., by preventing tissue from wrapping around components or elements of the device and/or by filling spaces within the device and thereby preventing tissue from penetrating such spaces). Coatings may also be provided to occlude flow of materials therethrough or through a structure to which the coating has been applied. Additionally or alternatively, the coatings may also provide a degree of structural stiffness to an otherwise more flexible device. Coatings may also be used to soften relatively sharp areas or structures of an implantable device. For instance, coatings may be used on angled corners of devices, or on free ends of elements such as filaments of woven materials (e.g., to protect welded ends of woven or braided filaments). It will be appreciated that reference may be made herein to terms such as filaments, strands, wires, fibers, struts, etc., interchangeably and without intent to limit.

In accordance with various further principles of the present disclosure, a coating is applied to at least a portion of the retention members which does not contact tissue at the deployment site. The coating may increase the pull out strength of the retention members. It will be appreciated that reference to increasing pull out strength may be understood as encompassing increasing retention strength, stiffness, resistance to migration (e.g., anti-migration properties), etc., of the otherwise flexible retention members. A coating, such as a biocompatible polymeric material, may also be applied to the ends of the implantable device, such as to coat welded ends of filaments (e.g., wires) braided or woven to form the implantable device.

In accordance with various principles of the present disclosure, to enhance the effect of the coating material on the pull out strength/retention strength of the retention members, at least one of the retention members is a double-wall retention member and the coating is applied to portions of the outer wall of the double-wall retention member which do not contact tissue. Optionally, a coating is applied to substantially all surfaces of one or both of the retention members which do not contact tissue. To further enhance the effect of the coating material, the surface area of the outer wall of the retention member to which the coating is applied may be increased in size relative to prior art retention members. For instance, an implantable device may be formed from a tubular element defining a lumen therethrough, and double wall retention members may be formed by extending the wall of the tubular element thereof radially outwardly to form the inner wall of a double-wall retention member, and then extending the wall of the tubular element radially inwardly from the perimeter of the inner wall of the retention member and towards the longitudinal axis of the tubular element to form the outer wall of the double-wall retention member. The surface area of an outer wall of a double wall retention member may be increased in accordance with various principles of the present disclosure by extending the outer wall closer to the central longitudinal axis of the implantable device than in prior devices. For instance, in some embodiments, the surface area may be increased by about 85% compared with previous similarly shaped (but not similarly sized) retention member outer walls. Previously, such further extension of the outer wall towards the longitudinal axis of the retention member had not been recognized as having potential benefits such as described herein. The present disclosure recognizes the previously unrecognized benefit of increasing the surface area of the outer wall of at least one of the retention members to increase the amount of coating material applied to such retention member, such as to increase pull out strength of the retention member. In some embodiments, the saddle outer diameter (which is smaller than the outer diameters of the retention members) is not constant, and, instead gradually increases from a narrow diameter generally at a central region thereof to transition to the larger outer diameter of one or both of the retention members. Such transition and gradual increase in diameter of the saddle may allow the saddle to more closely match the contours of the anatomy of the deployment site to increase contact of the saddle with tissue, and/or to increase the surface area of the saddle and thereby to improve tissue ingrowth in an uncoated saddle. Additionally or alternatively, such transition and gradual increase in diameter of the saddle may reduce fatigue on the material of the implantable device as it transitions from the saddle to one or both of the retention members. In some embodiments, the outer wall of at least one of the retention members is extended inwardly to an inner diameter substantially the same as (±approximately 0.5-0.1 mm and optionally ±approximately 0.1-2 mm, such measurements encompassing increments of 0.01 mm therebetween) the diameter of the saddle from which the retention member extends. For instance, the saddle may have a gradually increasing outer diameter to transition to the to the inner wall of the at least one retention member (e.g., at the point at which the wall of the implantable device extends substantially radially outwardly from and generally perpendicular to the longitudinal axis thereof to define a retention member). In some embodiments, the outer wall of the at least one retention member has a surface area of greater than about 500 mm², such as greater than about 750 mm², such as greater than about 1000 mm² (such as 1014 mm²), including increments of 2 mm² therebetween, resulting in an increase in surface area (compared to prior similar devices) over 100%, such as over 130% and even about 213%. Such increase in the surface area of the outer wall may be applied to either or both retention members, whether or not the lengths of the retention members (along the longitudinal axis of the implantable device) are substantially the same. Additionally or alternatively, the length of at least one of the retention members may be increased to increase the side surface area of the retention member which may contact tissue at the deployment site. In some embodiments, the increased length of at least one of the retention members may be achieved by increasing the distance between the inner wall and the outer wall thereof. In some embodiments, the increased length of at least one of the retention members may be in the form of an extension from the outer wall of the retention member.

In some aspects, tissue ingrowth into an implantable device, such as an implantable device formed in accordance with various principles of the present disclosure, is further induced by abrasion (e.g., mucosal abrasion) of the body tissue at the deployment site which is to be contacted by the implantable device. Different methods of abrasion may be performed, including, without limitation, use of a hook knife, hot biopsy forceps, hot snares, etc., to create abrasion of the tissue to elicit a healing response which would speed and increase the tissue ingrowth through the implantable device, thus reducing the likelihood of device migration. In accordance with various principles of the present disclosure, endoscopic mucosal resection (EMR) may be used in addition to or instead of mucosal abrasion. It has been discovered that EMR more completely removes the mucosa from the tissue to be contacted by the implantable device, and/or improves consistency in the method of enhancing tissue ingrowth into the implantable device. Such EMR technique, in combination with the modified configuration of an implantable device as described above has been shown in clinical studies to markedly reduce the incidence of migration of the implantable device from the deployment site. The EMR procedure may be performed with a hot snare, or other appropriate device, tool, instrument, etc., such as known to those of ordinary skill in the art, to cut and remove tissue from at least a selected region of the intended deployment site for the implantable device. A remaining region of tissue at the deployment site may be ablated using mucosal abrasion, or other appropriate techniques or procedures.

In accordance with an aspect of the present disclosure, the implantable device may be an occlusion device configured to regulate flow of material through an anatomical structure across which it is deployed, such as to occlude (understood herein to include fully or substantially fully or even partially occlude, unless otherwise indicated) flow of material therethrough. It will be appreciated that terms such as occlude, block, prevent, inhibit, impede, reduce, delay, etc. (and various grammatical forms thereof) may be used interchangeably herein without intent to limit to indicate reduction of flow of materials by greater than 50%, and up to 100% including increments of 1% therebetween. Reference may generally be made to an implantable device for the sake of convenience and without intent to limit, as it will be appreciated that various principles and aspects of the present disclosure are applicable to implantable devices other than those which specifically occlude flow (such, without limitation, devices holding tissues together in apposition, with or without forming an anastomosis therethrough) and/or which reduce flow of materials by less than 50%.

Various embodiments of implantable devices formed in accordance with various principles of the present disclosure will now be described with reference to examples illustrated in the accompanying drawings. Reference in this specification to “one embodiment,” “an embodiment,” “some embodiments”, “other embodiments”, etc. indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. Therefore, the present disclosure is not limited to only the embodiments specifically described herein, and the examples of embodiments disclosed herein are not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. It will be appreciated that, in the following description, elements or components similar among the various illustrated embodiments are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

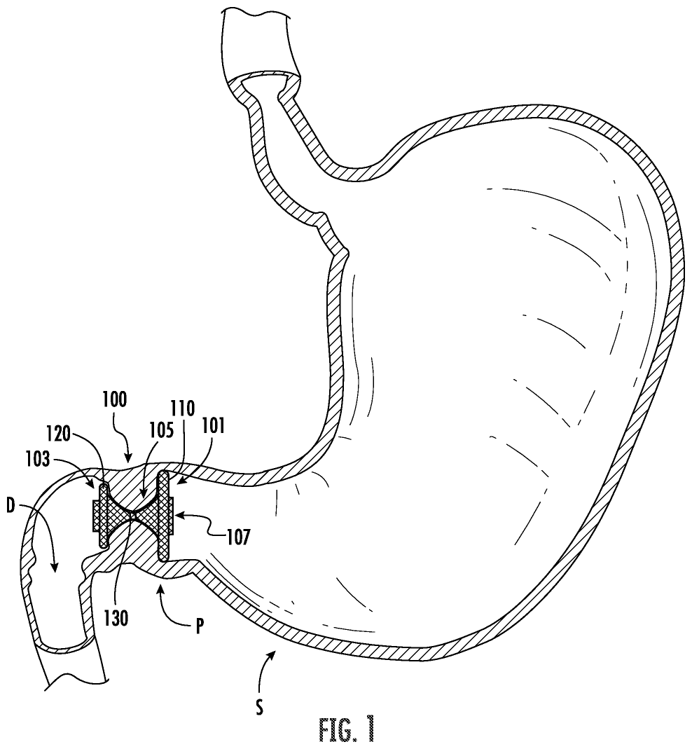

Turning now to the drawings, an example of an implantable device 100 formed in accordance with various principles of the present disclosure is illustrated in FIG. 1. The illustrated implantable device 100 has a proximal end 101, a distal end 103, and an intermediate region 105 therebetween. A proximal retention member 110 is provided along the proximal end 101 of the implantable device 100, and a distal retention member 120 is provided along the distal end 103 of the implantable device 100, with a saddle 130 extending between the proximal end 101 and the distal end 103 and along the intermediate region 105 of the implantable device 100. The saddle 130 may be configured to be positioned through a body passage, such as a pylorus P as illustrated in FIG. 1. The retention members 110, 120 are wider than the saddle 130, and are positioned against a respective body wall surrounding (e.g., extending outwardly from) a respective inlet and outlet of the body passage to prevent migration of the implantable device 100 with respect to the body passage (distally/proximally or downstream/upstream of the typical direction of flow of materials through the body passage). In the example of an environment illustrated in FIG. 1 for an implantable device 100 formed in accordance with various principles of the present disclosure, the proximal retention member 110 is positioned in the stomach S (such as against the antrum) and configured to inhibit distal migration of the implantable device 100 into the duodenum D, and the distal retention member 120 is positioned in the duodenum D and configured to inhibit proximal migration of the implantable device 100 into the stomach S. A lumen 107 may be defined through the implantable device 100 and may be partially or fully occluded, depending on the deployment site at which the implantable device 100 is to be deployed and/or the purpose of the implantable device 100 at the deployment site.

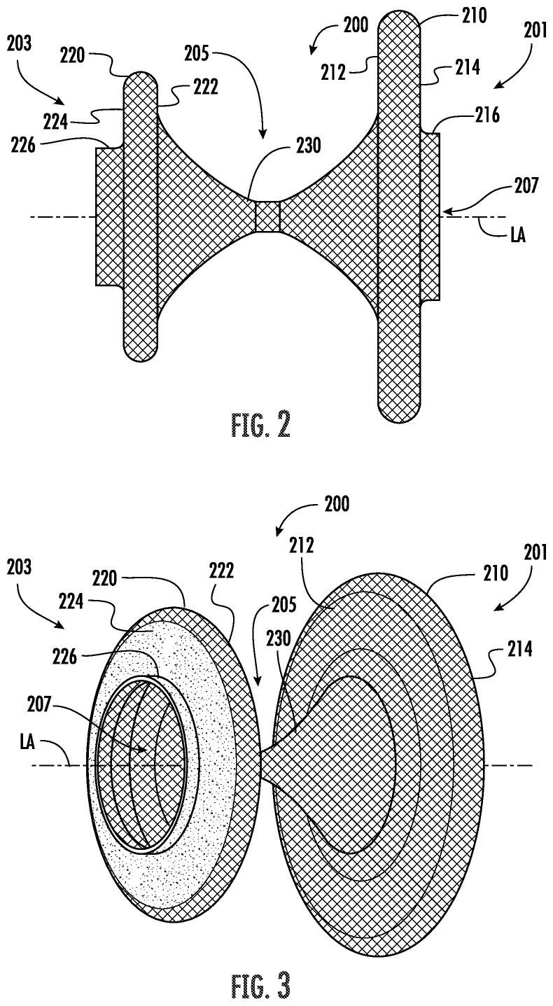

An example of an embodiment of an implantable device 200 formed in accordance with various principles of the present disclosure is illustrated in FIG. 2 and FIG. 3, having a first (e.g., proximal) retention member 210 along a first (e.g., proximal) end 201 thereof, a second (e.g., distal) retention member 220 along a second (e.g., distal) end 203 thereof, and a saddle 230 along an intermediate region 205 thereof. In accordance with various principles of the present disclosure, at least one of the retention members 210, 220 may be increased in diameter (in contrast with otherwise similar prior art devices) to increase the surface area thereof in contact with tissue at the deployment site. The illustrated example of an embodiment of an implantable device 200 may be configured for deployment across a pylorus P, such as illustrated in FIG. 1, The gastric retention member 210 may have a diameter larger than the diameter of the duodenal retention member 220, such as to expand further radially outwardly from the saddle 230 to increase the surface area in contact with the stomach S. Moreover, the gastric retention member 210 may have a diameter larger than the diameter of the duodenal retention member 220 (such as at least a 10% increase in diameter), and may be further increased to increase the surface area thereof in contact with at least the pyloric antrum of the stomach S. For instance, the diameter of the gastric retention member 210 may be about 30 mm, and even about 40 mm (including all increments between 30-40 mm), and even about 50 mm (including all increments between 40-50 mm) or larger.

In accordance with various principles of the present disclosure, surfaces of the implantable device 200 in contact with tissue at the deployment site are configured to promote tissue ingrowth along such surfaces. Coatings which may be applied for various purposes (such as to prevent passage of materials through the device wall, and/or to impart structural rigidity/decrease flexibility, and/or to impart a degree of lubriciousness such as to facilitate movement with respect to tissue or to prevent abrasion of tissue) may inhibit tissue ingrowth. In accordance with various principles of the present disclosure, such coatings are not provided on surfaces of the implantable device 200 in contact with tissue at the deployment site, thereby promoting tissue ingrowth into the uncoated surfaces. In some embodiments, surfaces configured to promote tissue ingrowth include all surfaces configured to contact tissue at the deployment site. In the example of an embodiment of an implantable device 200 illustrated in FIG. 2 and FIG. 3, the surface of the saddle 230 (a portion of the saddle 230 or optionally the entire surface of the saddle 230) is configured to promote tissue ingrowth. Additionally or alternatively, at least a portion of or all of the inwardly-facing sides 212, 222 of the retention members 210, 220, respectively, are configured to promote tissue ingrowth with respect to the retention members 210, 220. In the example of an embodiment of an occlusion device 200 illustrated in FIG. 2 and FIG. 3, the retention members 210, 220 are double-wall retention members such that the inwardly-facing sides 212, 222 of the retention members 210, 220 are inner walls of the double-wall retention members 210, 220, and the outwardly-facing sides 214, 224 of the retention members 210, 220 are outer walls of the double-wall retention members 210, 220. In some embodiments, all surfaces of the implantable device 200 configured to contact tissue at the deployment site are not coated with materials which may inhibit tissue ingrowth, or are otherwise configured to promote tissue ingrowth. Additionally or alternatively, such surfaces may be treated (e.g., coated) with materials which promote tissue ingrowth.

It will be appreciated that tissue ingrowth does not occur immediately upon deployment of the implantable device 200, and the implantable device 200 thus may be subject to migration or other undesirable shifting of position with respect to the deployment site until sufficient tissue has proliferated to secure the implantable device 200 with respect to the deployment site. In accordance with various principles of the present disclosure, a coating material is applied to selected portions of the occlusion device 200 to strengthen (e.g., increase rigidity/decrease flexibility) of the retention members 210, 220 and thereby to increase their pull out strength/resistance to migration. For instance, a coating material may be applied to surfaces of the outwardly-facing sides of the retention members 210, 220 which do not contact tissue at the deployment site. In the example of an embodiment of an implantable device 200 illustrated in FIG. 2 and FIG. 3, at least a portion of one or both of the respective outer walls 214,224 of the retention members 210, 220 is coated with a coating material. In some embodiments, the entire surface of one or both of the respective outer walls 214,224 of the retention members 210, 220 is coated with a coating material. Moreover, in some embodiments, such as illustrated in FIG. 2 and FIG. 3, one or both of the respective outer walls 214,224 of the retention members 210, 220 include axial extensions 216, 226, respectively, referenced herein simply as “lips” for the sake of convenience and without intent to limit. The lips 216, 226 extend away from the saddle 230 generally along the longitudinal axis LA of implantable device 200 with generally a smaller outer diameter than the outer diameter of the retention member 210, 220 from which it extends. The lips 216, 226 may serve various purposes, such as to be grasped for removal, and/or to facilitate finishing of the free ends of element from which the implantable device 200 has been formed and/or to add some stiffness to the retention member 210, 220 (especially if coated). As may be appreciated with reference to FIG. 2 and FIG. 3, in accordance with various principles of the present disclosure, some or all of the outer surface of one or both of the lips 216, 226 is coated with a coating material such as applied to the outer walls 214, 216 of the retention members 210, 220, such as to impart further structural rigidity to the implantable device 200.

In accordance with various principles of the present disclosure, the surface areas of the respective outer walls 214, 216 of the retention member 210, 220 are increased to be greater than what is typical for similar devices in the prior art. In other words, the diameter of the lips 216, 226 and/or the size of the portion of the lumen 207 extending through the outer walls 214, 216 is decreased relative to similar components of prior art devices (e.g., stents which typically have a maximum lip and lumen diameter throughout). An increase in the surface areas of the respective outer walls 214, 216 of the retention member 210, 220 allows for sufficient increase in coating material applied thereto to impart increased rigidity to the retention member 210, 220 to increase the respective pull out forces thereof. In some embodiments, an implantable device formed in accordance with various principles of the present disclosure provides about a 130% or greater increase in surface area (relative to current similar devices) that can be coated such as described herein.

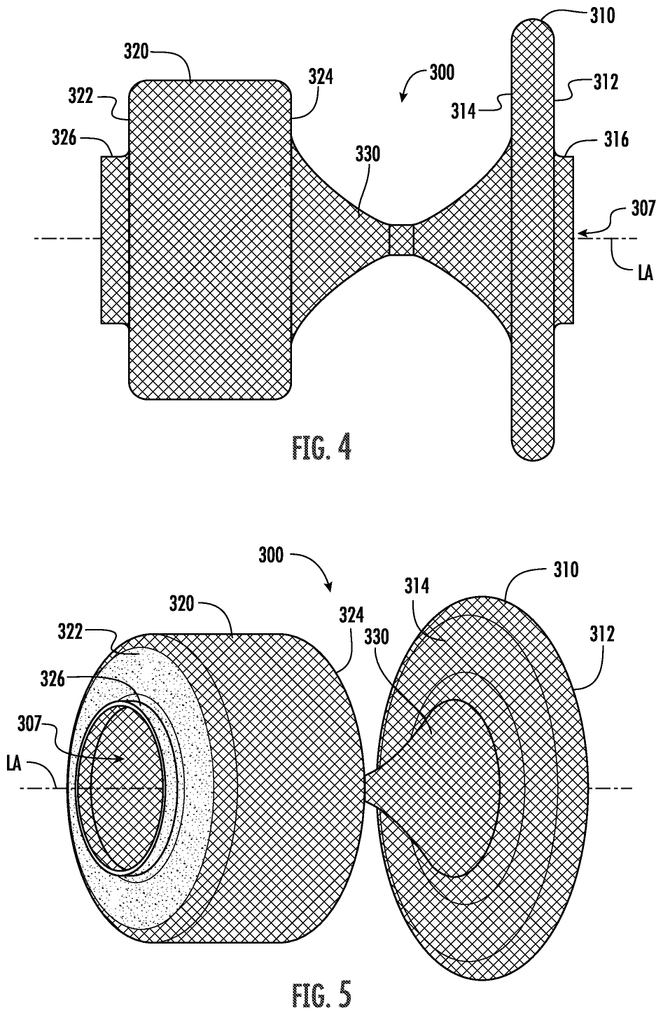

Various other modifications to the size, shape, configuration, and/or dimensions of an implantable device may be made in accordance with various principles of the present disclosure to resist migration of the device with respect to the deployment site. For instance, the surface area of surfaces of the implantable device configured to contact tissue may be increased and configured to promote tissue ingrowth (e.g., not coated with material which inhibits tissue ingrowth and/or coated with a material which promotes tissue ingrowth) to further reduce migration of the implantable device. In the example of an embodiment of an implantable device 300 illustrated in FIG. 4 and FIG. 5 the implantable device 300 is configured for deployment across a pylorus P, and the duodenal retention member 320 is increased in length to increase its surface area in contact with tissue wall of the duodenum in which it is deployed. As such, the implantable device 300 illustrated in FIG. 4 and FIG. 5 presents a greater amount of surface area for tissue ingrowth than presented by the corresponding retention members of the implantable device 200 illustrated in FIG. 2 and FIG. 3. The outer surface area of the outer walls 312, 322 of the retention members 310, 320 may be increased as described above and coated with a coating material to decrease the flexibility of the retention member 310, 320 and thereby to increase the pull out strength of the retention member 310, 320, as may be appreciated with reference to FIG. 5.

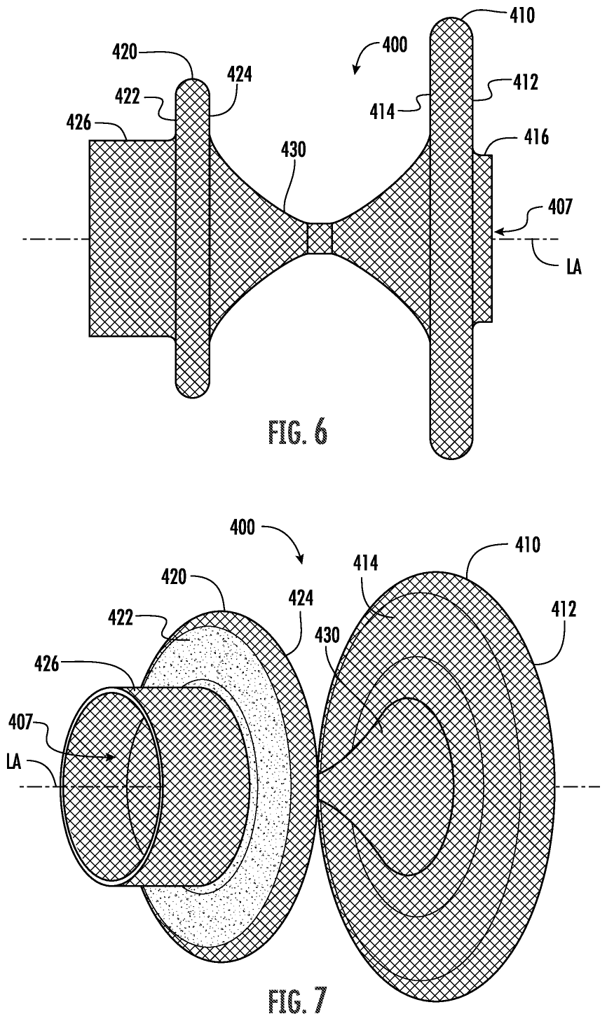

Additionally or alternatively, the length of the lip of at least one retention member of an implantable device 400 formed in accordance with various principles of the present disclosure may be extended to increase the surface area of an implantable device 400 for contact with tissue at a deployment site, such as illustrated in FIG. 6 and FIG. 7. The extended lip 426 advantageously may be configured to promote tissue ingrowth therein (e.g., not coated with material which inhibits tissue ingrowth and/or coated with a material which promotes tissue ingrowth). The implantable device 400 illustrated in FIG. 6 and FIG. 7 with an extended length-of lip 226 may be suitable for deployment in a pylorus (such as illustrated in FIG. 1) to increase contact of a region of the implantable device 400 (the lip 426 extending from the duodenal retention member 420) with the duodenum. The present disclosure recognizes increased rigidity may be imparted by proximity of the walls of a double-wall retention member. In accordance with various principles of the present disclosure, instead of extending the length of a retention member (distance between the inner wall and the outer wall thereof) to increase the surface area of the retention member in contact with tissue at the deployment site, the inner wall 422 and the outer wall 424 of the duodenal retention member 420 of the implantable device 400 illustrated in FIG. 4 and FIG. 5 are closer together than the inner wall 322 and the outer wall 324 of the duodenal retention member 320 of the occlusion device 300 illustrated in FIG. 4 and FIG. 5 to increase rigidity of the retention member 420.

An implantable device with one or more features as described above may be constructed in a variety of non-limiting manners. In some embodiments, an implantable device with one or more features as described herein may be formed from a tubular element. In some embodiments, an implantable device formed in accordance with various principles of the present disclosure is formed from a plurality of filaments which are formed into a tubular element. It will be appreciated that the term filament is used for the sake of convenience, and may be used interchangeably herein with such terms as wires, strands, fibers, struts, etc., without intent to limit. In some embodiments, the implantable device is formed from a plurality of filaments braided, woven, interwoven, knitted, wrapped, intertwined, looped (e.g., bobbinet-style), knotted, or otherwise formed into a tubular element. In some embodiments, the implantable device is formed from a laser-cut tube, which may in some instances be considered to form a plurality of struts forming a tubular element. In some embodiments, the implantable device is formed from a plurality of bonded elongated elements. An implantable device formed in accordance with various principles of the present disclosure may be formed of a biocompatible metal or a polymeric material or an alloy or a combination thereof. In other examples, the implantable device may be constructed from a polymeric material (e.g., polyethylene terephthalate, poly(methyl methacrylate)). In yet other examples, the implantable device may be constructed from a combination of metallic and polymeric materials. In still yet other examples, the implantable device may include a bioabsorbable and/or biodegradable material (e.g., a poly(lactic-co-glycolic acid) polymer).

Formation of the walls of an implantable device as described above may leave a plurality of openings therethrough, such as between the filaments or struts or the like. It will be appreciated that the term openings is used for the sake of convenience, and may be used interchangeably herein with such terms as spaces or interstices or the like without intent to limit. Deployment of such walls in contact with tissue may allow or promote tissue to grow into the interstices of the walls, thereby securing the walls with respect to the deployment site.

To promote tissue ingrowth into an implantable device, tissue at the deployment site may be pretreated in a variety of manners, such as by abrasion, ablation, pharmaceutical treatments, argon plasma coagulation (APC), etc., to promote, accelerate, and/or increase cellular growth, such as a result of a healing response. Various forms of abrasion, including use of a hook knife, hot biopsy forceps, and hot snares, may be used to create abrasion of tissue such as mucosal tissue of the gastrointestinal tract, such as in the region of the pylorus. In accordance with various principles of the present disclosure, improved inducement of tissue growth may be achieved by performing endoscopic mucosal resection (EMR) on tissue at the deployment site of an implantable device. In some embodiments, the tissue is blebbed (such as by injection of a fluid, such as a sterile saline solution); the blebbed tissue is pulled (such as with a vacuum); and the pulled tissue is resected (such as with a hot snare or blade or other cutting instrument) to separate the mucosal tissue from the submucosal tissue at the resection site. For example, EMR may advantageously be performed on tissue surrounding and/or along the pylorus. Such technique more completely removes the mucosa from the tissue to be contacted by the implantable device and improves the consistency of the tissue-growth-promoting method performed prior to deployment of the implantable device. Generally, EMR is a more consistent technique than prior techniques for increasing tissue growth as EMR allows a more readily definable (and also generally greater) amount of tissue to be grasped and removed, such action inducing a healing response with the desired accompanying tissue growth.

Although, for anti-migration purposes, it is desirable to promote tissue ingrowth and to leave surfaces of an implantable device formed in accordance with various principles of the present disclosure free of coatings which may inhibit tissue ingrowth, it will be appreciated that application of a coating to regions of an implantable device may be desirable to occlude flow of materials through interstices in the walls of the device. Additionally or alternatively, application of a coating to walls of an implantable device may strengthen the walls such as by filling interstices in the walls, thereby reducing the flexibility of the coated region and strengthening such region of the device wall. Such coatings may be formed from a biocompatible polymeric material, such as silicone, urethane, polyurethane, urethane polyether block amides (PEBA), polyethylene, polyethylene terephthalate (PET), polyvinylidene difluoride (PVDF), polytetrafluoroethylene (PTFE), expanded polytetrafluoroethylene (ePTFE), polyester, polypropylene, polynapthalene, Chronoflex®, C-Flex® thermoplastic elastomer, Krator® SEBS and SBS polymers, or similar biocompatible polymeric formulations and/or copolymers thereof and/or combinations thereof. Additional or other features or structures (e.g., barbs or looped projections) may be provided to promote tissue ingrowth in accordance with various principles of the present disclosure.

As discussed above, the surface area of retention members of an implantable device formed in accordance with various principles of the present disclosure may be increased to increase the available surface area to be coated with a material which increases the pull out strength of the coated retention member. Such enlarged retention members may be formed in a variety of manners. For instance, the material of the tubular element (e.g., the material of filaments forming a braided or wire mesh tubular element) from which an implantable device such as disclosed herein is formed may be a shape-memory and/or heat formable material, such as, without limitation, a nickel-titanium alloy (e.g., Nitinol) or a Cobalt-Chromium-Nickel-Molybdenum alloy (e.g., Elgiloy®), or stainless steel. The configuration of the tubular element may be set by shaping the tubular element such as by compressing or constraining or constricting or clamping or otherwise narrowing an intermediate region (e.g., with a shape-forming tool, such as a mold form) to form a saddle, and optionally expanding the end regions to form retention members extending radially outwardly along one or both ends of the implantable device. In accordance with various principles of the present disclosure, at least one of the free ends of the tubular element from which the implantable device is formed are drawn inwards to a greater extent than in similar prior art devices to increase the surface area of the outer walls of the retention member adjacent such free end. The present disclosure recognizes the previously unrecognized benefit of increasing the surface area of the outer walls of the retention members and decreasing the diameter of any lumen opening at the ends of the implantable device and/or the diameter of the lips of the implantable device such as to provide increased surface area for receiving a coating to increase the overall pull out force of the retention members. The thus formed implantable device may be treated, in a manner known to those of ordinary skill in the art (e.g., heated), to set the formed tubular element into the desired shape the details of which do not form a part of the present disclosure.

To facilitate delivery and deployment and optional removal endoscopically or transluminally (or otherwise, without the need for open surgery), an implantable device formed in accordance with various principles of the present disclosure may be configured to shift or move between a collapsed or compact delivery configuration and an expanded deployment configuration. In some embodiments, the implantable device is formed to facilitate such ability to shift configurations. In some embodiments, the implantable device is formed of a material which may be advantageously used to facilitate expansion of the device into a desired deployment configuration, such as a self-expanding and/or shape memory material. The implantable device may be configured to be returned to a collapsed or compact configuration to facilitate removal from the deployment site as desired and/or medically indicated. The shape or configuration or general construction of the device may facilitate shifting to a collapsed or compact configuration in any of a variety of known or heretofore known manners, the precise construction not being critical to the broad principles of the present disclosure.

It should be understood that, as described herein, an “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more embodiments of concepts or features together in a single embodiment of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one embodiment can be used separately, or with one or more other features to yield a still further embodiment. Thus, it is intended that the present subject matter covers such modifications and variations as come within the scope of the appended claims and their equivalents.

In view of the above, it should be understood that the various embodiments illustrated in the figures have several separate and independent features, which each, at least alone, has unique benefits which are desirable for, yet not critical to, the presently disclosed implantable device. Therefore, the various separate features described herein need not all be present in order to achieve at least some of the desired characteristics and/or benefits described herein. Only one of the various features may be present in an implantable device formed in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure. Moreover, throughout the present disclosure, reference numbers are used to indicate a generic element or feature of the disclosed embodiment. The same reference number may be used to indicate elements or features that are not identical in form, shape, structure, etc., yet which provide similar functions or benefits. Additional reference characters (such as letters, as opposed to numbers) may be used to differentiate similar elements or features from one another.

Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An implantable device extending along a longitudinal axis and having a first end, a second end, and an intermediate region therebetween, said implantable device comprising:
a saddle along said intermediate region;
a first retention member extending radially outwardly from the longitudinal axis along said first end; and
a second retention member extending radially outwardly from the longitudinal axis along said second end;
wherein:
at least a portion of the surface of said saddle and at least a portion of inwardly-facing surfaces of said first retention member and said second retention member facing towards said saddle are configured to promote tissue ingrowth therewith; and
outwardly-facing sides of said first retention member and said second retention member facing away from said saddle are sized such that coating material provided thereon increases the pull out strength of said first retention member and said second retention member.

2. The implantable device of claim 1, wherein the outwardly-facing side of at least one of the first retention member or the second retention member has a surface area of greater than about 500 mm².

3. The implantable device of claim 1, wherein a lip extends axially from said second retention member a greater distance than the distance between the inwardly-facing side and the outwardly-facing side of said second retention member and is configured to promote tissue growth therein.

4. The implantable device of claim 1, wherein:
said first retention member and said second retention member each comprise a double wall retention member with an inner wall along the inwardly-facing side thereof and an outer wall along the outwardly-facing side thereof; and
the distance between said inner wall and said outer wall of said second retention member is greater than the distance between said inner wall and said outer wall of said first retention member.

5. The implantable device of claim 4, further comprising a lip axially extending from at least one of said first retention member or said second retention member in a direction away from said saddle, wherein said lip is coated with a coating material reducing flexibility thereof.

6. The implantable device of claim 1, wherein said first retention member and said second retention member are different sizes, shapes, configurations, and/or dimensions.

7. The implantable device of claim 1, wherein:
said saddle is configured to be positioned through a pylorus;
said first retention member is configured to be seated in a portion of a stomach surrounding the pylorus; and
said second retention member is configured to extend into a duodenum extending from the pylorus.

8. The implantable device of claim 7, wherein said saddle is narrowed to occlude flow of materials through the pylorus.

9. The implantable device of claim 7, wherein the outer diameter of said first retention member is greater than the outer diameter of said second retention member.

10. The implantable device of claim 1, wherein said saddle, and sides of said first retention member and said second retention member facing inwardly towards said saddle have walls with interstices defined therein and are left uncoated to promote tissue ingrowth into the interstices.

11. An implantable device formed from a tubular element having a first end, a second end, an intermediate region therebetween, and a lumen defined therethrough, said implantable device comprising:
a saddle along the intermediate region;
a first retention member defined by a portion of said tubular element along the first end of said tubular element having a first diameter larger than the outer diameter of said saddle to form a first retention member inner wall facing said saddle and a first retention member outer wall facing away from said saddle; and
a second retention member defined by a portion of said tubular element along the second end of said tubular element having a second diameter larger than the outer diameter of said saddle to form a second retention member inner wall facing said saddle and a second retention member outer wall facing away from said saddle;
wherein said first free end of said tubular element has a diameter sufficiently smaller than the outer diameter of the first retention member to provide sufficient surface area to receive a coating material to increase the rigidity of said first retention member outer wall.

12. The implantable device of claim 11, wherein the diameter of at least one of the first free end of said tubular element and the second free end of said tubular element is smaller than the largest diameter of said saddle.

13. The implantable device of claim 11, wherein said retention members have different sizes, shapes, configurations, and/or dimensions.

14. The implantable device of claim 13, wherein the outer diameter of said first retention member is larger than the outer diameter of said second retention member.

15. The implantable device of claim 13, wherein the distance between said second retention member inner wall and said second retention member outer wall is greater than the distance between said first retention member inner wall and said first retention member outer wall.

16. The implantable device of claim 13, further comprising a lip extending axially from said second retention member away from said saddle, and having a length greater than the distance between said second retention member inner wall and said second retention member outer wall.

17. The implantable device of claim 16, wherein said second retention member outer wall is coated and the outer surface of said lip is configured to promote tissue ingrowth.

18. A method of deploying an implantable device at a deployment site, said method comprising:
performing endoscopic mucosal resection of a portion of tissue at the deployment site so that tissue growth is promoted at the resected tissue site; and
deploying the implantable device with a surface contacting the resected tissue site configured to allow tissue ingrowth therein.

19. The method of claim 18, ablating tissue at the deployment site surrounding the resected tissue using mucosal abrasion.

20. The method of claim 18, further comprising:
deploying a saddle, defined along an intermediate region of the implantable device, across a body passage;
deploying a first retention member against tissue surrounding an inlet into the body passage; and
deploying a second retention member against tissue surrounding an outlet of the body passage;
wherein the first retention member has an outer diameter greater than the outer diameter of the second retention member and has an inwardly-facing side contacting the tissue surrounding the inlet and configured to promote tissue ingrowth therein, and an outwardly-facing side facing away from the body passage and not contacting tissue and coated with a material increasing the pull out strength thereof, the outer diameter of the first retention member and the material coated thereon increasing the retention strength of the first retention member against migration of the implantable device into the body passage.
